(19) United States
(12) Plant Patent Application Publication
(10) Pub. No.: US 2003/0088899 P1
(43) Pub. Date: May 8, 2003

(54) COLEUS PLANT NAMED 'SUMCOL 01'

(76) Inventor: Ralf Schrader, Ludinghausen (DE)

Correspondence Address: Russell D. Orkin, Attorney for Applicant, 700 Koppers Building, 436 Seventh Avenue, Pittsburgh, PA, 15219-1818, US

(21) Appl. No.: 10/053,418

(22) Filed: Nov. 7, 2001

(51) Int. Cl. A01H 5/00

(52) U.S. Cl. PLT/373

(57) ABSTRACT

A Coleus plant named ‘Sumcol 01’ particularly characterized by its strong fragrance and compact, rounded shape. The plant has an appealing leaf color. The plant has shown resistance to botrytis, snails, and caterpillars. Animals avoid the present plant because of its strong fragrance.

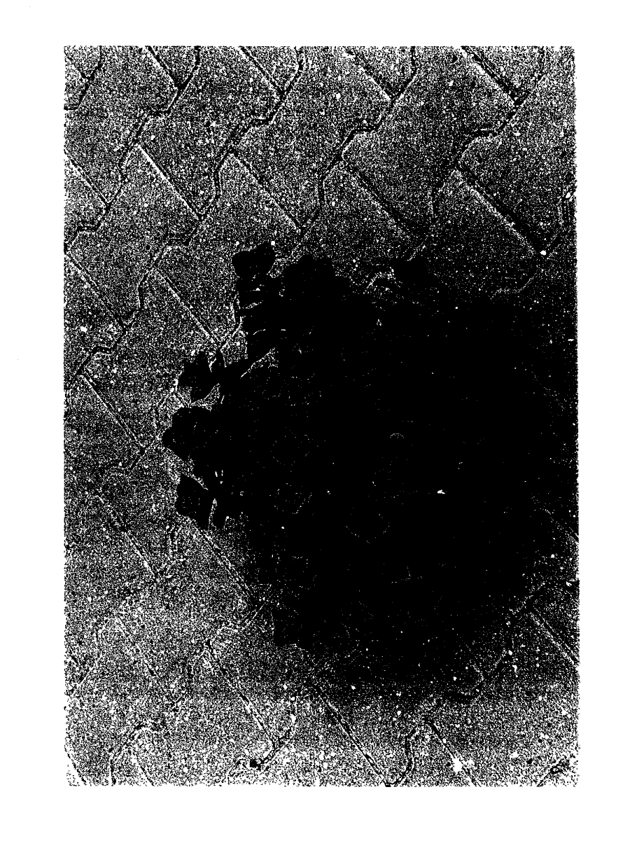

### BACKGROUND OF THE NEW PLANT

The present invention comprises a new and distinct cultivar of Coleus canina hybridus known by the varietal name ‘Sumcol 01’.

The new cultivar was discovered in a planned breeding program in Lüdinghausen Germany in July 2000 and is a controlled cross of an unnamed, unpatented female Coleus canina plant with an unnamed, unpatented male Coleus esculentus plant. The purpose of the breeding program was to produce a Coleus plant with compact growth habit.

The following traits distinguish the new cultivar from other cultivated plants of this type known to the inventor. The characteristics are described with comparative reference to the cultivar's female parent.

1. The female parent grows stronger than ‘Sumcol 01’ and is in hanging form.

2. ‘Sumcol 01’ has a stronger fragrance than the female parent.

3. ‘Sumcol 01’ has better leaf color than the female parent.

4. ‘Sumcol 01’ is more compact than the female parent.

The first asexual reproduction took place at Lüdinghausen, Germany in January 2001 by cuttings. Continued observations from the vegetative cuttings have confirmed that the distinguishing features of the new cultivar remain stable and are retained through successive propagations.

### DESCRIPTION OF THE DRAWING

The accompanying photographic drawing illustrates the new cultivar, the color being as nearly true as possible with color illustrations of this type.

### DESCRIPTION OF THE NEW PLANT

The following detailed description sets forth the characteristics of the new cultivar. The data which defines these characteristics were collected from asexual reproductions carried out in Lüdinghausen, Germany. Color references are primarily to the R.H.S. Colour Chart of The Royal Horticultural Society of London except where general terms of ordinary dictionary significance were used.

### PLANT

Classification: Coleus canina hybridus.

Height: 25 cm.

Width: 30 cm.

Form: Compact, rounded.

Stems:

Size.—21 mm long.

Surface texture.—Fleshy.

Color.—Yellow-Green Group 153A.

Branches:

Surface texture.—Fleshy.

New growth color.—Yellow-Green Group 145A.

Mature growth color.—Yellow-Green Group 146C.

Internode length.—10.5 mm.

Leaves:

Length.—35 mm.

Width.—33 mm.

Surface texture.—Rippled.

Shape.—Obovate.

Color.—Upper surface: Greyed-Green Group 189A. Lower surface: Greyed-Green Group 191A.

Mid-vein.—Size: 17 mm. Color: Yellow-Green Group 145C.

Petioles.—

Length.—12 mm.

Thickness.—Moderate.

Color.—Yellow-Green Group 145C.

Growing conditions: The new cultivar is preferably cultivated in peat soil at a pH between 5.5 and 6.0 not subject to freezing. The natural flowering period is summer.

Disease resistance: Botrytis; snails; caterpillars.

Other features: Cats and dogs avoid the present cultivar because of its strong fragrance.

### INFLORESCENCE

Peduncles:

Color.—Yellow-Green Group 145A.

Length.—80 mm.

### BUDS

Shape: Spike.

Length: 40 mm.

Color: Yellow-Green Group 147A.

### FLOWERS

Average number of blooms per cluster: 20 to 30.

Form: Single to bilabiate.

Color when opening begins:

Upper surface.—Violet-Blue Group 97B.

Lower surface.—Violet-Blue Group 97B.

Color when blooming:

Upper surface.—Violet-Blue Group 97B.

Lower surface.—Violet-Blue Group 97B.

Color at end of blooming:

Upper surface.—Violet-Blue Group 97B.

Lower surface.—Violet-Blue Group 97B.

Fragrance: Unpleasant.

Petal form: Two different forms: Ovial and labiate.

Petal number: Two.

Lasting quality: 5 to 8 days from opening to end of life.

Sexual characteristics: Bisexual.

### FRUIT

Maturity date: 21 days from crossing until fruit produced.

Size: 0.5 mm, uniform.

Color: Brown Group 200A.

Flesh texture: Hard.

I claim:

1. A new and distinct variety of coleus plant as herein shown and described, particularly characterized by its strong fragrance and compact, rounded shape.
